(19) United States
(12) Patent Application Publication
(10) Pub. No.: US 2015/0257751 A1
(43) Pub. Date: Sep. 17, 2015

(54) SUTURING DEVICE

(71) Applicant: SATURIX LTD., Tel Aviv (IL)

(72) Inventors: Yehuda Bachar, Givaat Shmuel (IL); Zvi Goltzman, Rosh Ha'ayin (IL)

(21) Appl. No.: 14/423,500

(22) PCT Filed: Sep. 2, 2013
(86) PCT No.: PCT/IB2013/058231

§ 371 Date: Feb. 24, 2015

Related U.S. Application Data
(60) Provisional application No. 61/696,186, filed on Sep. 2, 2012.

(51) Int. Cl. A61B 17/04

(52) U.S. Cl. CPC A61B 17/0469; A61B 17/0401; A61B 17/0482; A61B 2017/0409

(57) ABSTRACT

Apparatus for securing a suture thread to a region of tissue, the apparatus comprising: first and second elongate jaws respectively having first and second longitudinal axes and first and second gripping surfaces for gripping and holding a region of tissue at a tilt angle relative to the first and second axes when the jaws are closed; a channel in the first elongate jaw having a substantially straight section extending along the first longitudinal axis and a curved section that extends from the straight section to the first gripping surface for housing at least one suture anchor configured to secure a portion of a suture thread to the tissue region, and; a flexible needle inside the channel that extends along the straight section and is controllable to extract a suture anchor in the channel from the first jaw through the curved section and drive the extracted suture anchor through the tissue region to secure the suture thread.

### RELATED APPLICATIONS

The present application claims the benefit under 35 U.S.C. 119(e) of U.S. Provisional Application 61/696,186 filed on Sep. 2, 2012, the disclosure of which is incorporated herein by reference

### TECHNICAL FIELD

Embodiments of the invention relate to a suturing device suitable for use in minimal invasive surgical procedures.

### BACKGROUND

Minimal invasive surgical (MIS) procedures refer to procedures that are performed on an internal organ and/or structure, hereinafter a “target organ”, of a patient's body through small openings, often referred to as “keyholes” or “ports”, made in the skin and outer wall of the body. The procedures are conventionally referred to by a body cavity in which the target organs they are designed to treat are located. For example, MIS procedures performed on target organs in the abdominal cavity are referred to as laparoscopic procedures. If performed on a target organ in the chest, a MIS procedure may be referred to as a thoracoscopic procedure. MIS procedures performed on target organs of a joint are referred to as arthroscopic procedures.

Access via a keyhole to an internal operating site at which a target organ of an MIS procedure is located, is facilitated by an access tube or tube arrangement, hereinafter referred to generically as an “access tube”, introduced into the body through the keyhole. An access tube typically comprises a cannula, trocar, and/or trocar and cannula combination, and generally has a relatively small cross-section lumen having a diameter in a range between about 5 mm and about 12 mm. The access tube is oriented so that it extends towards the operating site with a distal, open end of the access tube positioned inside the body at a location, generally near the operating site, convenient for performance of the MIS procedure. The access tube may be used to introduce into the body various MIS surgical devices used in performance of the MIS procedure so that the surgical devices have appropriate access to the target organ at the site.

The MIS surgical devices generally comprise a long, small cross section shaft configured to be introduced into the access tube and have at a distal end of the shaft a tool to be used at the operating site. The tools typically introduced into a patient's body via an access tube comprise components of MIS imaging systems for viewing target organs or surgical tools for manipulating target organ tissue. Imaging system components that are introduced into the body through an access tube may include video cameras, components of endoscopes, illumination systems, ultrasound imaging systems and miniature MRI imaging devices. Surgical tools introduced into the body through an access tube may include, scissors, scalpels, tissue ablation devices, grabbers, and suturing tools.

The imaging devices and surgical instruments introduced into the body through an access tube, and in particular suturing tools, often prove difficult to control with advantageous dexterity, whether controlled by a human or robotic surgeon. Typically, the MIS surgical devices require a surgeon to exhibit a relatively high degree of hand-eye coordination, which may be acquired generally only after repeated practice.

### SUMMARY

An aspect of an embodiment of the invention relates to providing an MIS suturing device for securing a suture thread to a layer of tissue inside a body with a plurality of suture anchors that are attached to the suture thread and which the suturing device drives through the tissue layer.

An aspect of an embodiment of the invention relates to providing suture anchors that exhibit relatively small resistance to penetrating and being driven through a layer of tissue together with a portion of the suture thread to be secured to the tissue, but once driven through the layer of tissue may exhibit enhanced resistance to being pulled out of the tissue.

An aspect of an embodiment of the invention relates to providing the suturing device with a delivery system for positioning the suture anchor at the tissue layer and driving the suture anchor and the portion of suture thread through the tissue layer to secure the suture thread to the tissue layer.

In an embodiment of the invention, the suture anchors are “torpedo” shaped comprising an elongate body having a long axis and tapered front end. The torpedo shape of a suture anchor in accordance with an embodiment of the invention provides relatively low resistance to being driven through a layer of tissue, tapered end first, and with its long axis substantially perpendicular to the tissue layer. Once driven through the tissue layer, the suture anchor may be rotated so that the long axis is substantially parallel to the tissue layer. With its long axis substantially parallel to the tissue layer, the suture anchor and a portion of the suture thread attached to the anchor exhibit relatively large resistance to being pulled back out through the tissue layer.

In an embodiment of the invention the suturing device comprises a set of first and second opposable elongate alligator jaws which may be closed to grip and clamp a region of the tissue layer through which a suture anchor is to be driven between respective gripping surfaces of the jaws. The gripping surfaces are located at distal ends of the elongate jaws and optionally are planar and substantially parallel when the jaws are closed. The jaws are mounted to a support barrel configured to deliver the alligator jaws to a tissue region through a trocar.

The first jaw is formed having a channel that houses a needle, hereinafter a drive needle, for driving the suture anchors through the region of tissue clamped between the respective gripping surfaces of the first and second alligator jaws. The drive needle passes through each of the plurality of suture anchors along the long axes of the anchors, and the drive needle together with the plurality of suture anchors through which it passes are housed in the channel. The channel comprises a relatively long straight section that extends substantially the length of the jaw. The gripping surface of the first jaw is tilted with respect to a direction along which the straight section extends. A relatively short curved section of the channel extends from the straight section to meet the gripping surface substantially at 90°.

The suturing device is controllable to shuttle the needle in and out of the channel to transport suture anchors one by one from the channel to exit the first jaw through the gripping surface of the first jaw and drive the anchor through the tissue region. The second jaw is formed having a recess that opens at the gripping surface of the second jaw for receiving the suture anchor and its associated portion of the suture thread that the drive needle drives through the tissue region. In transporting the suture anchors from the channel, the drive needle and the suture anchor that it transports bend when moving through the curved section and the needle and the anchors are configured to elastically deform to mediate the bend.

Because the curved section of the channel is substantially perpendicular to the gripping region of the first and second jaws, the drive needle drives the suture anchor through the tissue region substantially parallel to the axis of the anchor along a direction substantially perpendicular to the tissue region. In an embodiment of the invention, the suture anchor is configured to be rotated by tension applied to the suture thread after the anchor is driven through the tissue region and the suturing device tensions the suture thread to rotate the suture anchor after it has been driven through the tissue region to orient the axis of the anchor substantially parallel to the tissue region. The recess in the second jaw may comprise a one way anchor stop that operates to prevent the suture anchor that it receives from being pulled back through the tissue region by tension generated in the suture thread by the suturing device and aid in rotating the suture anchor.

In the discussion, unless otherwise stated, adjectives such as “substantially” and “about” modifying a condition or relationship characteristic of a feature or features of an embodiment of the invention, are understood to mean that the condition or characteristic is defined to within tolerances that are acceptable for operation of the embodiment for an application for which it is intended. Unless otherwise indicated, the word “or” in the description and claims is considered to be the inclusive “or” rather than the exclusive or, and indicates at least one of, or any combination of items it conjoins.

This Summary is provided to introduce a selection of concepts in a simplified form that are further described below in the Detailed Description. This Summary is not intended to identify key features or essential features of the claimed subject matter, nor is it intended to be used to limit the scope of the claimed subject matter.

### BRIEF DESCRIPTION OF FIGURES

Non-limiting examples of embodiments of the invention are described below with reference to figures attached hereto that are listed following this paragraph. Identical features that appear in more than one figure are generally labeled with a same label in all the figures in which they appear. A label labeling an icon representing a given feature of an embodiment of the invention in a figure may be used to reference the given feature. Dimensions of features shown in the figures are chosen for convenience and clarity of presentation and are not necessarily shown to scale.

FIGS. 1A-1J schematically illustrate a suturing procedure using suture anchors to secure a length of suture thread to opposite tissue edges of a tear in a tissue to sew the edges together, in accordance with an embodiment of the invention;

FIG. 2 schematically shows a simplified side view of a suturing device operable to perform a suturing procedure similar to that illustrated in FIGS. 1A-1J, in accordance with an embodiment of the invention;

FIG. 3A schematically shows alligator jaws and components of the alligator jaws comprised in the suturing device shown in FIG. 2 that are used to grab and hold a layer of tissue and drive a suture anchor through the tissue, in accordance with an embodiment of the invention;

FIG. 3B schematically shows the alligator jaws and internal components of the alligator jaws shown in FIG. 2A clamped to a region of tissue prior to driving a suture anchor through the tissue, in accordance with an embodiment of the invention;

FIG. 3C schematically shows the alligator jaws and internal components of the alligator jaws shown in FIG. 2C clamped to a region of tissue after driving a suture anchor and a portion of suture thread through the tissue, in accordance with an embodiment of the invention;

FIG. 3D schematically shows an enlarged cross section view of components of the alligator jaw used to regulate driving suture anchors one by one through a region of tissue, in accordance with an embodiment of the invention; and

FIGS. 4A-8C schematically illustrate control and operation of a suturing device in driving a suture anchor through a region of tissue in accordance with an embodiment of the invention.

### DETAILED DESCRIPTION

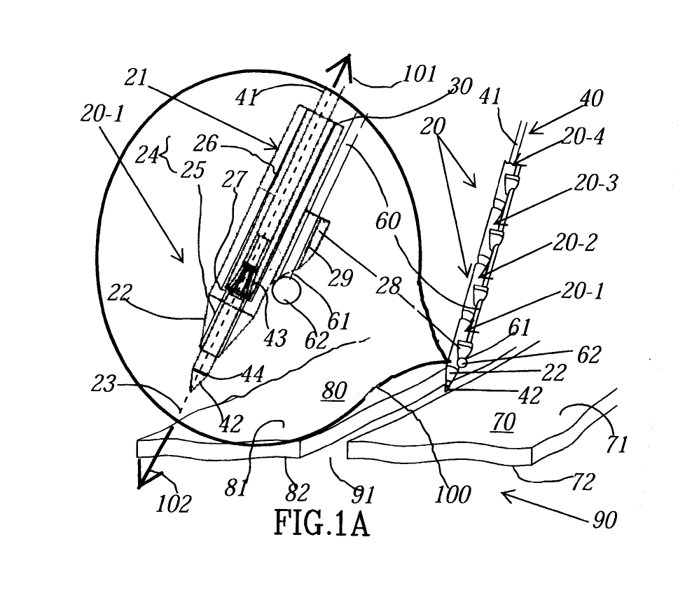

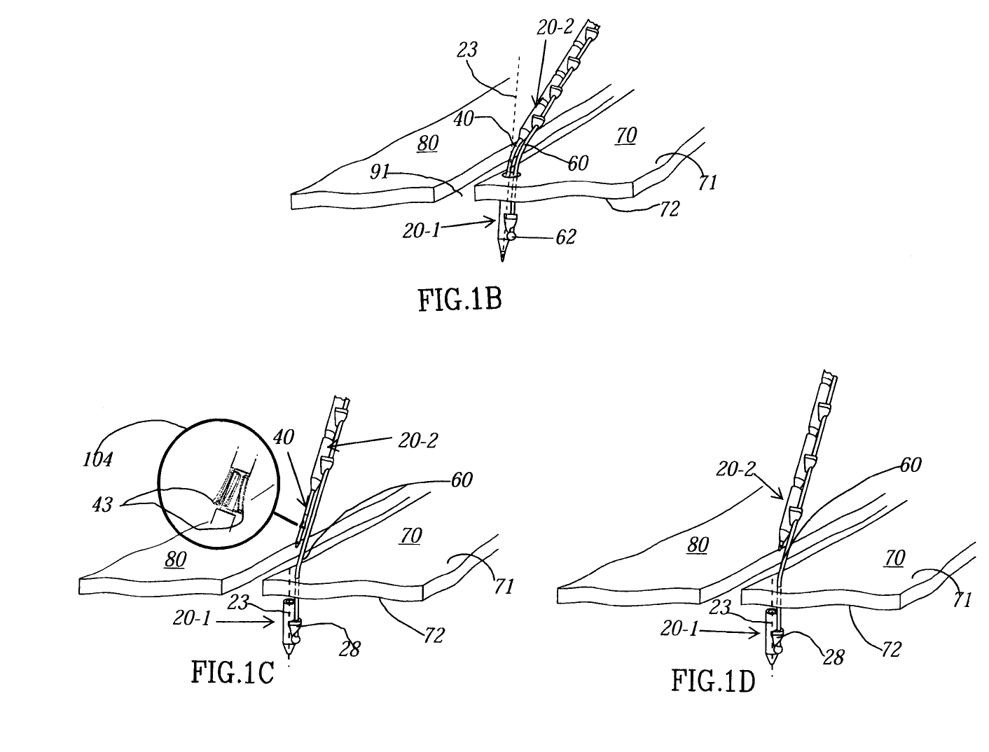

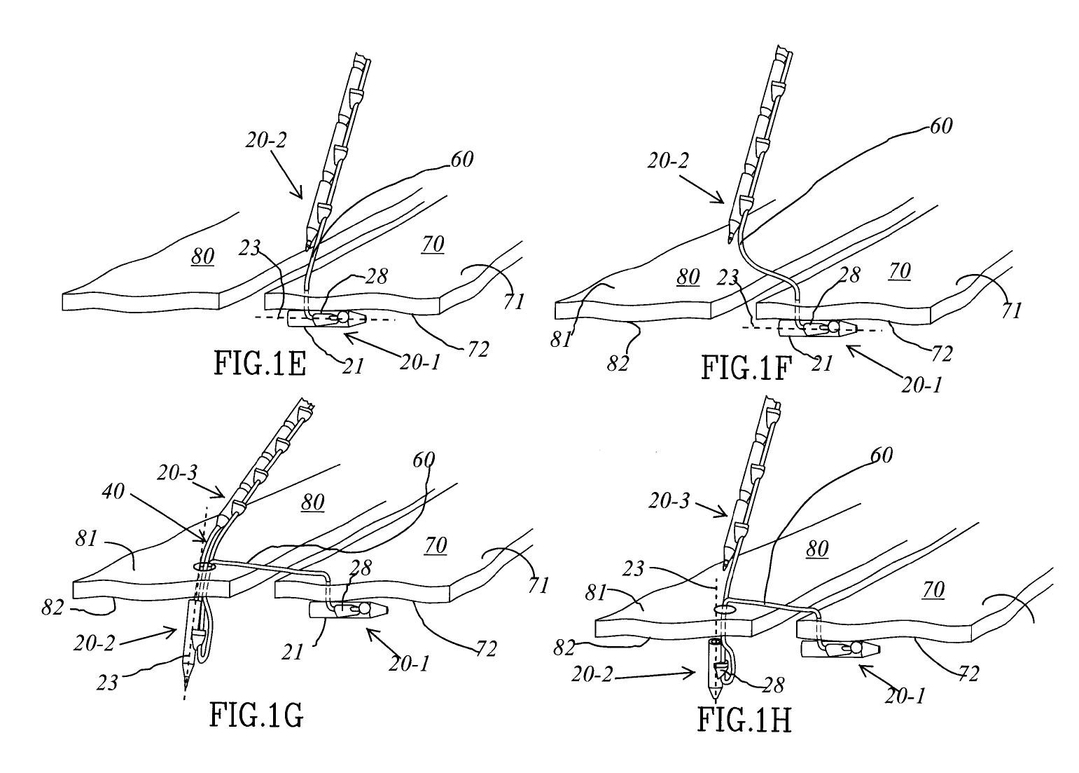

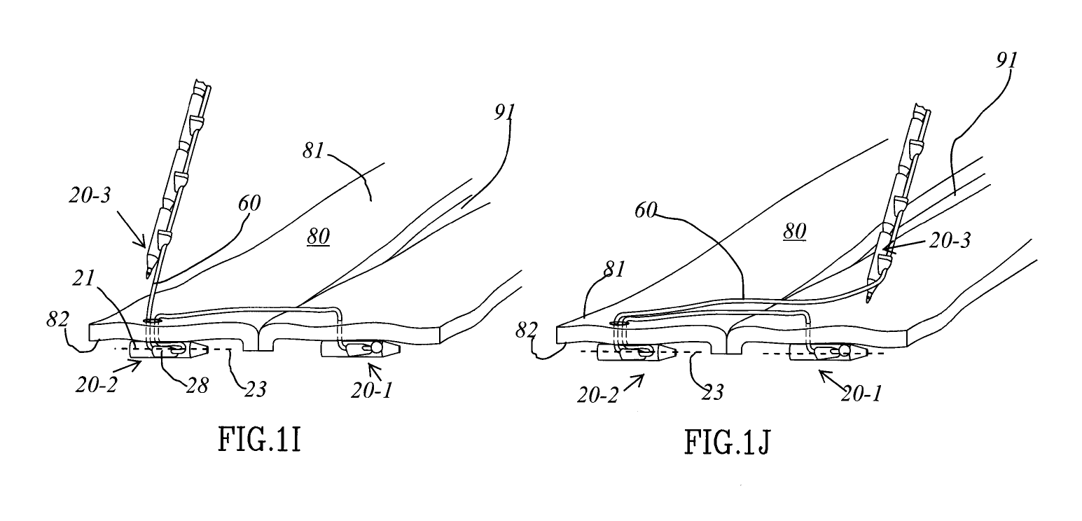

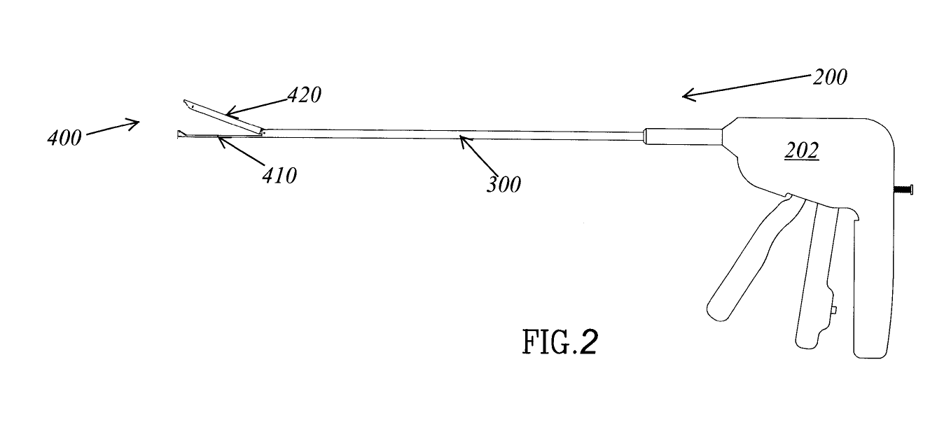

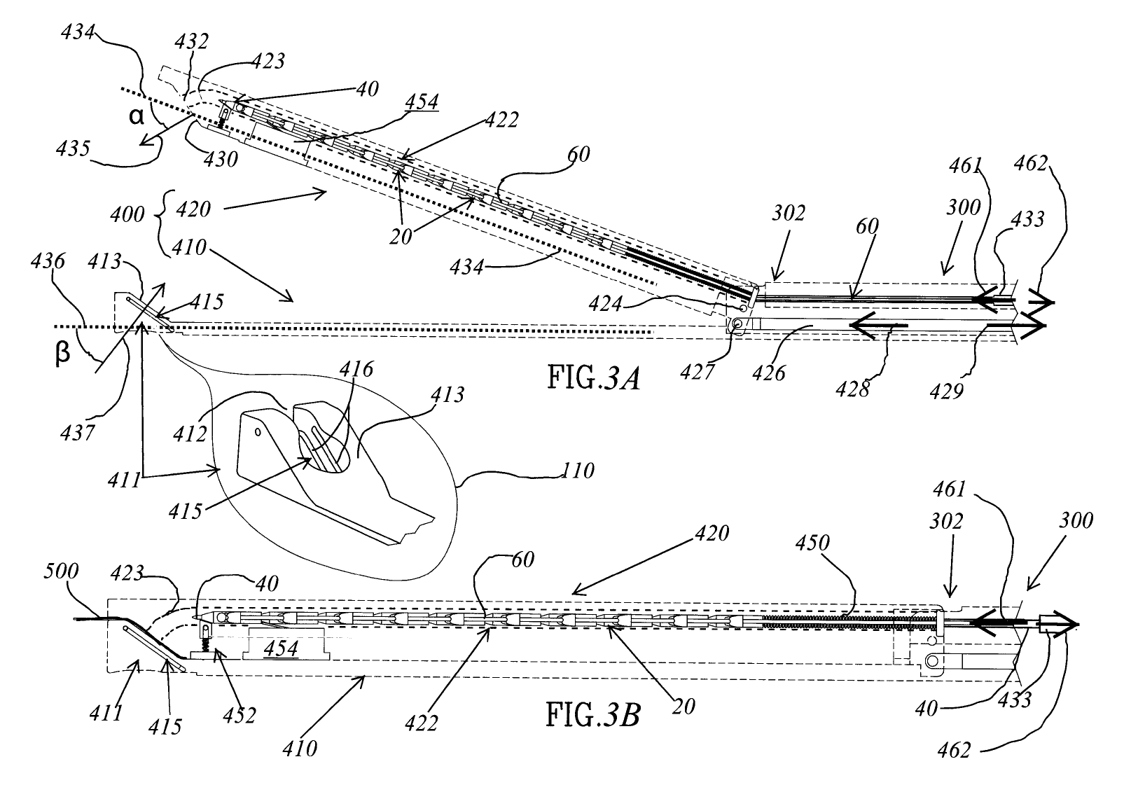

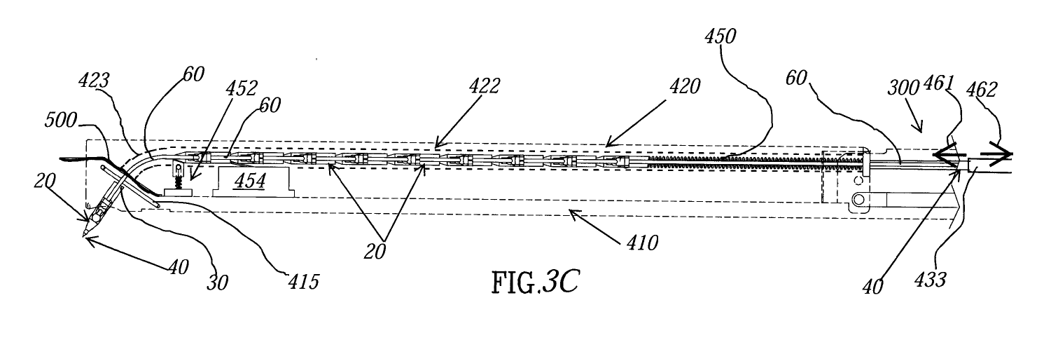

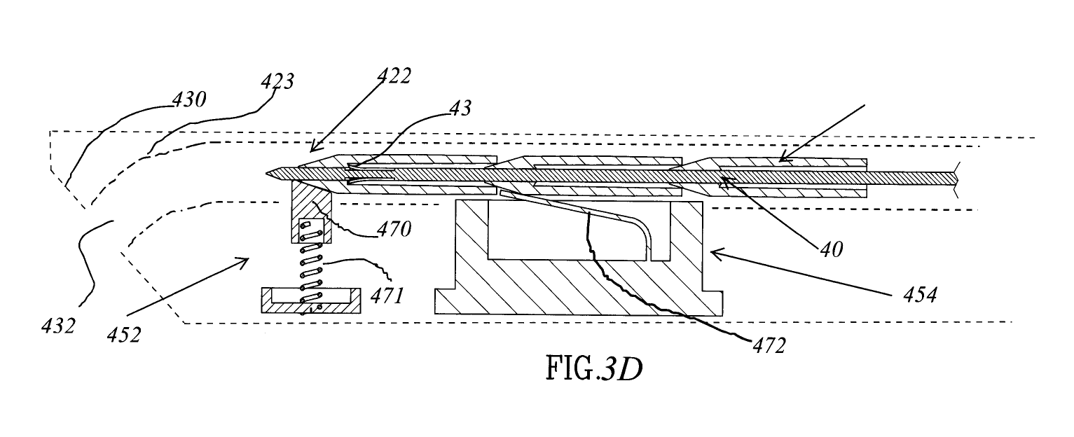

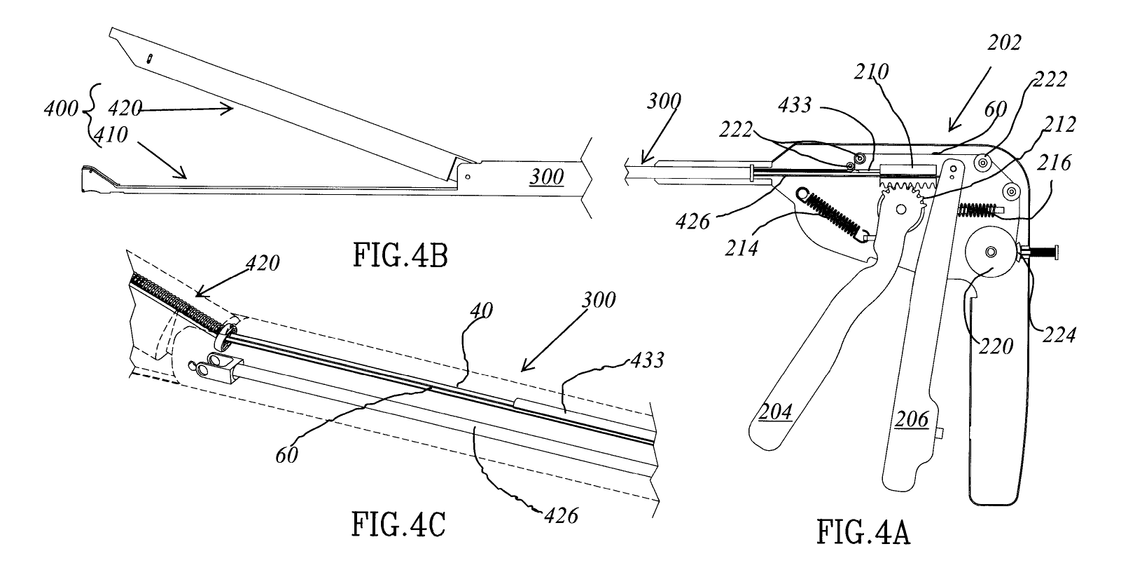

In the detailed description below the kinematics of a procedure for suturing a layer of tissue to close a tear in the tissue layer, in accordance with an embodiment of the invention are discussed with reference to FIGS. 1A-1J. The figures illustrate puncturing the tissue, one by one with each of a plurality of suture anchors mounted to a same flexible drive needle to secure a suture thread to the tissue and close the tear in accordance with an embodiment of the invention. A suturing device for implementing a procedure similar to that shown in FIGS. 1A-1J is shown in FIG. 2 and discussed with reference to the figure. Features of the suturing device shown in FIG. 2 and mechanical components optionally comprised in a suturing device in accordance with an embodiment of the invention, are shown in FIGS. 3A-3F and discussed with reference to the figures. Operation of the suturing device and cooperation of its mechanical parts to implement a procedure similar to that shown in FIGS. 1A-1J are discussed with reference to FIGS. 4A-8C.

FIGS. 1A-1J schematically illustrate using suture anchors 20 mounted to a flexible drive needle 40 that passes through the suture anchors to secure a length of suture thread 60 to opposite tissue regions 70 and 80 of a tear 91 in a tissue layer 90 to sew the opposite tissue regions together, in accordance with an embodiment of the invention. Tissue region 70 has top and bottom surfaces 71 and 72 respectively and tissue region 80 has top and bottom surfaces 81 and 82 respectively. A side of tissue region 70 facing top surface 71 may be referred to as a top side 71 of the tissue region and a side of tissue region 70 may be referred to as a bottom side 72 of the tissue region. Similarly, top and bottom sides of tissue region 80 may be referred to as top and bottom sides 81 and 82 respectively.

FIG. 1A shows a plurality of suture anchors 20 threaded, butted front to back, on drive needle 40 poised above top surface 71 of tissue region 70 immediately prior to driving a first suture anchor 20, referred to as a “lead” suture anchor and distinguished by a reference numeral 20-1, through tissue region 70. Suture anchors 20, located behind lead suture anchor 20-1 are distinguished by numerals 20-2, 20-3 . . . 20-N, where the number following the dash in each numeral corresponds to a position behind lead suture anchor 20-1 of the suture anchor that the numeral distinguishes. “N” is a number of the plurality of suture anchors mounted to drive needle 40. An enlarged cross section view of lead suture anchor 20-1 is shown in an inset 100.

Suture anchors 20 optionally comprise an elongate torpedo shaped hollow body 21 having a tapered front end 22 a non-tapered back end 30, a long axis 23, and a lumen 24 through which drive needle 40 passes parallel to the long axis. Body 21 may have any of various external cylindrical shapes. Body 21 may for example be circularly cylindrical or elliptically cylindrical. Axis 23 and lumen 24 are shown for lead suture anchor 20-1 in inset 100. Lumen 24 is optionally formed as a two chamber lumen having a small cross section front chamber 25 and a larger cross section back chamber 26. An annular ridge 27 is formed where chambers 25 and 26 meet. Each suture anchor 20 optionally comprises a fin 28 attached to anchor body 21. Fin 28 is formed having a hole 29 through which suture thread 60 passes.

Except for possibly lead suture anchor 20-1, suture thread 60 is free to slide in hole 29 of fin 28. Optionally, a lead end 61 of suture thread 60 is formed having an anchor guard, schematically represented by a sphere 62, that prevents lead suture anchor 20-1 from sliding off the suture thread. By way of example, anchor guard 62 may comprise a knot tied in lead end 61 of suture thread 60 or a bead formed at the lead end. The bead may be formed by heating lead end 61 of suture thread 60 to melt material in the lead end so that it forms a bead. In an embodiment of the invention lead suture anchor 20-1 may be fixed to lead end 61 of suture thread 60, for example by crimping fin 28 to the suture thread, to prevent the lead suture anchor from sliding off suture thread 60.

Drive needle 40 has a shaft 41 formed from a suitable elastic material such as stainless steel or nitinol and a pointed tip 42 suitable for puncturing tissue. Optionally, drive needle 40 comprises at least one elastic “splay spur” 43 which is configured to splay out elastically away from shaft 41 so that an end of the at least one splay spur facing toward tip 42 of drive needle 40 displaces away from the shaft. At least one splay spur 43 may be drawn into large cross section chamber 26 of lumen 24 through small chamber 25 by translation of drive needle 40 along axis 23 “backwards” in a direction indicated by an arrow 101 in inset 100, pointing away from tapered front end 22 of suture anchor 20-1. Once drawn into large cross section chamber 26, at least one splay spur 43 may splay out elastically to “catch” on ridge 27. When at least one splay spur 43 is caught on ridge 27, translation of drive needle shaft 41 along axis 23 in a direction towards tapered end 22 of lead suture anchor 20-1 couples the suture anchor to drive needle shaft 41 so that the suture anchor translates with the needle shaft. Optionally, drive needle 40 has a raised lip 44 near tip 42 of the drive needle that operates to prevent a suture anchor 20 from easily sliding forward off the tip of the drive needle.

In FIG. 1B drive needle 40 is pushed “forward” carrying with it lead suture anchor 20-1, which is locked to the drive needle by at least one splay spur 43 (FIG. 1A), to penetrate and drive the lead suture anchor through tissue region 70. In driving lead suture anchor 20-1 through tissue region 70, flexible drive needle 40 bends so that the lead suture anchor may be driven through tissue region 70 at an advantageous “penetration angle”. In an embodiment of the invention, suture anchors 20 are configured to bend with drive needle 40 when the drive needle bends to drive the suture anchor through a region of tissue.

Penetration angle of a suture anchor 20 being driven through a tissue region may be an angle that axis 23 of the suture anchor makes with a plane of the tissue region. Optionally, the plane of the tissue region is a surface of the tissue region that the suture anchor first breaches when penetrating the tissue region. In FIG. 1B penetration angle of suture anchor 20-1 is an angle that axis 23 of suture anchor 20-1 makes with surface 71 of tissue region 70. In an embodiment of the invention the penetration angle is advantageously equal to about 90° so that in FIG. 1B axis 23 is substantially perpendicular to surface 71. Because lead end 61 of suture thread 60 comprises anchor guard 62, or lead suture anchor 20-1 is fixed to the suture thread, when lead suture anchor 20-1 is driven through tissue region 70 the lead suture anchor carries with it a portion of suture thread 60.

Methods and apparatus for pushing drive needle 40 forward to carry a single suture anchor, such as lead suture anchor 20-1, of the plurality of suture anchors 20 mounted to drive needle 40 and bend drive needle 40 to orient a suture anchor 20 at an advantageous penetration angle are discussed below.

Following penetration of lead suture anchor 20-1 through tissue region 70, drive needle 40 is retracted out of the lead suture anchor and out of tissue region 70 to leave lead suture anchor 20-1 on bottom side 72 of the tissue region and couple to next suture anchor 20-2 threaded on drive needle 40. FIG. 1C schematically shows drive needle 40 retracted from lead suture anchor 20-1 and tissue region 70 before it is sufficiently retracted to couple at least one splay spur 43, and thereby the drive needle, to suture anchor 20-2. In the retracted state shown in FIG. 1C at least one splay spur 43 is visible and shown greatly enlarged in an inset 104. FIG. 1D schematically shows drive needle 40 after it has been sufficiently retracted to couple to suture anchor 20-2.

As noted above, raised lip 44 (FIG. 1A) prevents a suture anchor 20, such as lead suture anchor 20-1, from easily sliding off tip 42 of the drive needle. However, an amount by which lip 44 is raised is not sufficient to prevent drive needle 40 from being retracted from a suture anchor 20 after the suture anchor has been driven through a tissue region by a suturing device in accordance with an embodiment of the invention. In an embodiment of the invention, the suturing device, as discussed below, comprises a “one-way anchor stop” that operates to prevent a suture anchor from being pulled back through a tissue region by retraction of drive needle 40 after the suture anchor has been driven through the tissue region.

Following retraction of drive needle 40 as shown in FIG. 1D, suture thread 60 is drawn taut. Drawing the suture thread taut applies force to fin 28, which because the fin is located on a side of lead suture anchor 20-1, generates a torque. The torque operates to rotate lead suture anchor 20-1 and orient axis 23 parallel to bottom surface 72 of tissue region 70 and press elongate body 21 of the suture anchor substantially parallel to the bottom surface as schematically shown in FIG. 1E. With lead suture anchor 20-1 rotated parallel and pressed to bottom surface 72, lead suture anchor 20-1 is relatively difficult to dislodge and pull out through tissue region 70 and the lead suture anchor securely holds a portion of suture thread 60 on bottom side 72 of tissue region 70.

After securing a portion of suture thread 60 on bottom side 72 of tissue region 70, drive needle 40 is positioned over tissue region 80 as schematically shown in FIG. 1F to puncture tissue region 80 and drive suture anchor 20-2 together with a portion of suture thread 60 attached to suture anchor 20-2 through the tissue region. FIG. 1G schematically shows suture anchor 20-2 after it has been driven through tissue region 80. Following driving suture anchor 20-2 through tissue region 80, drive needle 40 is retracted to couple to suture anchor 20-3 as schematically shown in FIG. 1H and suture thread 60 is drawn taut to rotate suture anchor 20-2 and secure the suture anchor pressed to bottom surface 82 of tissue region 80. FIG. 1I schematically shows suture anchor 20-2 after it has been rotated and pressed to bottom surface 82.

Because suture thread 60 is free to slide through fin 28 of suture anchor 20-2, drawing suture thread 60 taut draws tissue region 80 to tissue region 70 and closes a portion of tear 91 in the vicinity of suture anchors 20-1 and 20-2 as schematically shown in FIG. 1I. Drive needle 40 is then optionally repositioned over tissue region 70 as schematically shown in FIG. 1J to drive suture anchor 20-3 through tissue region 70 and secure another portion of suture thread 60 to tissue region 70. Drive needle 40 is then optionally repositioned over tissue region 80 to drive suture anchor 20-4 through the tissue region, draw taut suture thread 60 and close another portion of tear 91. The process is repeated, alternately driving a suture anchor 20 and a portion of suture thread 60 attached to the suture anchor through tissue region 70 and tissue region 80 and drawing the suture thread taut to close additional regions of tear 91 until the tear is closed and/or a last suture anchor 20 is secured to tissue region 70 or tissue region 80. Following driving a last suture anchor 20 (optionally a suture anchor 20-N) into one of tissue regions 70 or 80 suture thread 60 is optionally tied to the tissue regions using a conventional knotting procedure.

FIG. 2 schematically shows a simplified side view of a suturing device 200 configured to perform a suturing procedure similar to that shown in FIGS. 1A-1J in accordance with an embodiment of the invention. Suturing device 200 optionally comprises a manually operated control handle 202 connected to a relatively long thin support barrel 300 comprising a pair 400 of opposable alligator jaws 410 and 420, collectively referred to as alligator jaws 400. Opposable alligator jaws 410 and 420 are controllable by operation of control handle 202 to grab and hold a layer of tissue and drive a suture anchor 20 from alligator jaw 420 through the tissue layer to alligator jaw 410, in accordance with an embodiment of the invention. Support barrel 300 is dimensioned to be inserted into an MIS access tube (not shown) to position alligator jaws 400 at an internal site in a patient's body where they are to be used. Handle 202 optionally has first and second triggers 204 and 206 that are operated to control functions of suturing device 200. Details of control handle 202 and control of functions of suturing device 200 are discussed below with reference to FIGS. 4A-8C.

FIG. 3A schematically shows an enlarged view of alligator jaws 410 and 420 and a portion of support barrel 300 near a distal end 302 of the support barrel to which the jaws are connected. Dashed lines are used to indicate features which are to be considered transparent to show internal components of the features or components housed in the features.

Alligator jaw 420, also referred to as a “drive needle jaw 420”, is formed having a suture anchor feed channel 422 that houses drive needle 40 and suture anchors 20 shown in FIGS. 1A-1I, mounted on the drive needle. Suture anchor feed channel 422 extends the length of drive needle jaw 420 to a surface 430 at which the anchor feed channel has an opening 43 and may have any suitable cross section that accommodates drive needle 40 and suture anchors 20. For example, suture anchor feed channel may have a substantially circular, elliptical, tear or egg shaped cross section. Drive needle 40, is controllable to shuttle back and forth in anchor feed channel 422 into and out of drive needle jaw 420 through opening 432 of surface 430 to carry suture anchors 20 one by one together with portions of suture thread 60 to which they are coupled out of drive needle jaw 420 and drive the anchors through a tissue layer. A drive needle push rod 433 connected to the drive needle and housed in support barrel 300 is controlled by control handle 202 (FIG. 2) to control motion of drive needle 40.

In an embodiment of the invention, drive needle face 430 is tilted with respect to a longitudinal axis represented by a dotted line 434 of drive needle jaw 420, and suture anchor feed channel 422 comprises a curved section 423 that meets opening 432 on needle face surface 430. An angle, hereinafter a “tilt angle” of drive needle face 430 may be defined by an acute angle α that a normal, indicated by an arrow 435, to surface 430 makes with longitudinal axis 434. At opening 432 a tangent (not shown) to curved section 423 is substantially perpendicular to drive needle face 430. As a result, when drive needle 40 shuttles back and forth to sequentially drive suture anchors 20 through a region of tissue it exits and enters drive needle jaw 420 substantially perpendicular to drive needle face 430. To negotiate curved section 423 of suture anchor feed channel 422, drive needle 40 and suture anchors that the drive needle transports through curved section 423 are configured in accordance with an embodiment of the invention to resiliently bend at a minimum radius of curvature that characterizes curved section 423.

By way of a non-limiting numerical example, in an embodiment of the invention, drive needle 40 may have a circularly cylindrical body 41 having a diameter between about 0.15 and about 0.6 mm and may be formed from a resilient material such as stainless steel or nitinol. In an embodiment of the invention body 41 has a diameter equal to about 0.25 mm.

Body 21 of a suture anchor 20 is optionally circularly cylindrical, having an outer diameter between about 0.3 mm and about 1.5 mm and length from about 2.5 mm to about 7 mm. In an embodiment of the invention, body 21 has a diameter equal to about 0.85 mm and length equal to about 4 mm. For a 0.85 mm diameter body, fin 28 may protrude about 0.5 mm from the body. Small chamber 25 of lumen 24 is optionally circular having a diameter between about 0.1 and about 0.6 mm. In an embodiment of the invention, chamber 25 has a diameter equal to about 0.24 mm. Large chamber 26 is optionally circular having a diameter between about 0.3 mm and about 1.3 mm. In an embodiment of the invention large chamber 26 has a diameter equal to about 0.58 mm. A suture anchor 20 in accordance with an embodiment of the invention may be formed from a resilient material such as Teflon, or an elastomer, such as polyurethane, styrene-ethylene-butadiene-styrene (SEBS), or silicon.

Suture anchor feed channel 422 has any cross section shape suitable for accommodating drive needle 40 and suture anchors 20. In an embodiment of the invention the cross section has a maximum dimension between about 0.8 mm and about 2.5 mm. To accommodate a suture anchor having a circularly cylindrical body 21 and fin 28 protruding from a side of the body, suture anchor channel 422 may have a “Russian doll” shape formed substantially by a perimeter around a shape formed by a large circle and a small circle intersecting. To accommodate a suture anchor body having diameter 0.85 mm and fin 28 protruding 0.45 mm the large and small circles may have diameters equal to about 0.9 and 0.7 mm and a maximum dimension along a line joining the centers of the circles equal to about 1.33 mm. Curved section 423 of channel 422 may have a minimum radius of curvature between about 2.5 mm and about 4.5 mm. In an embodiment of the invention curved section has a radius of curvature equal to about 3.5 mm. Optionally, angle α is greater than or equal to about 30°. In an embodiment of the invention, a is greater than or equal to about 45°. Optionally a is greater than or equal to about 60°.

In an embodiment of the invention, alligator jaws 400, when closed can be passed through a trocar having an internal diameter less than or about equal to 12 mm. Optionally the closed alligator jaws are passable through a trocar having an internal diameter less than or about equal to 9 mm. In an embodiment, closed alligator jaws 400 can be passed through a trocar having an internal diameter less than or equal to about 5 mm.

Alligator jaw 410, also referred to as backstop jaw 410, is optionally a rigid extension of support barrel 300 and operates as a “backstop” to drive needle jaw 420 when alligator jaws 410 and 420 are closed to clamp a region of tissue being sutured using suturing device 200 and to counter forces generated by driving a suture anchor 20 from drive needle jaw 420 through the tissue region.

Drive needle jaw 420 is rotatably connected to support barrel 300 so that it may be rotated away from and towards backstop jaw 410 about a swivel pin 424 at distal end 302 of support barrel 300 to open and close alligator jaws 410 and 420 and grab and clamp a tissue layer through which drive needle 40, drives a suture anchor 20. A push rod 426 housed in support barrel 300 controls position of drive needle jaw 420. Push rod 426 is optionally connected to drive needle jaw 420 by a swivel pin 427 and translation of the push rod in directions indicated by arrows 428 and 429 respectively rotates drive needle jaw 420 away from and toward backstop jaw 410. Push rod 426 is controlled by operation of control handle 202, details of which are discussed below.

FIG. 3B schematically shows alligator jaws 410 and 420 closed and grabbing a tissue layer 500 so that drive needle 40 can drive a suture anchor 20 with its associated portion of suture thread 60 from needle drive jaw 420 through the tissue layer.

Backstop jaw 410 optionally comprises an anchor backstop 411 having a recess 412, and a backstop surface 413. Recess 412 is shown in an enlarged perspective view of anchor backstop 411 in an inset 110. Backstop surface 413 is tilted by a tilt angle 13 with respect to a longitudinal axis 436 of backstop jaw 410. Tilt angle 13 is an acute angle that a normal, represented by an arrow 437 to backstop surface 413 makes with longitudinal axis 436. Optionally, tilt angle 13 is substantially equal to tilt angle α.

When alligator jaws 410 and 420 are closed to grab and hold a region of tissue through which suturing device 200 (FIG. 2) drives a suture anchor 20 and its associated portion of suture thread 60, the tissue region is clamped between backstop surface 413 and drive needle face surface 430, and suture anchor 20 is received in recess 412 of anchor backstop 411. Backstop surface 413 and drive needle face surface 430 may be referred to as “gripping” or “clamping” surfaces. In an embodiment of the invention, anchor backstop 411 comprises a one way anchor stop that operates to prevent the suture anchor that it receives in recess 412 from being pulled back through the tissue region by retraction (schematically shown in, and discussed above with reference to FIGS. 1C and 1D) of drive needle 40 after the suture anchor has been driven through the tissue region.

FIG. 3C schematically shows tissue layer 500 clamped between backstop surface 413 and drive needle surface 430 of alligator jaws 410 and 420 after a suture anchor 20 has been driven through tissue layer 500 and before drive needle 40 is retracted from the suture anchor and the tissue layer.

In an embodiment of the invention, a one way anchor stop comprises a spring clip 415 through which a suture anchor 20 passes after being driven through the tissue region. The spring clip has arms 416 that are elastically pushed apart by the tapered front end 22 and body of suture anchor 20 during passage of the suture anchor through the spring clip. After the suture anchor has passed through spring clip 415, arms 416 snap back and “lock” behind the non-tapered back end 30 (FIG. 1A) of the suture anchor to prevent the suture anchor from being pulled out of the tissue through the spring clip.

By tilting drive needle face surface 430 and backstop surface 413, providing feed channel 422 with curved section 423 and configuring, drive needle 40 and suture anchors 20 to negotiate the curved section, alligator jaws 410 and 420 may be made relatively long up to 7 cm to enable convenient gripping of a region of tissue and driving a suture anchor 20 through the region substantially perpendicular to the tissue. In particular, the relatively long alligator jaws 410 and 420 may enable gripping and suturing a tissue region using suture anchors 20 at relatively large distances from an edge of the tissue.

Shuttling drive needle 40 in and out of drive needle jaw 420 along feed channel 422 to pick up and drive suture anchors 20 one by one through a tissue layer is controlled by drive needle push bar 433 (FIG. 3A, FIG. 3C) and by a feed spring 450 (FIGS. 3B-3C), and front and back anchor stops 452 and 454 housed in drive needle jaw 420. A schematic enlarged cross section view of front and back anchor stops 452 and 454 is shown in FIG. 3D together with drive needle 40 coupled to a suture anchor 20 by splay spurs 43.

Push bar 433 is connected to control handle 202 and the control handle is operated to move the push bar back and forth in directions indicated by arrows 461 and 462. Moving push bar 433 back and forth in directions indicate by arrows 461 and 462 shuttles drive needle respectively out from and back into drive needle jaw 420. Feed spring 450 spring loads suture anchors 20 so that as each suture anchor 20 is removed from feed channel 422 by motion of drive needle 40 out from the feed channel to be driven through a layer of tissue, the remaining suture anchors 20 are pushed forward along drive needle 40 until, as schematically shown in FIG. 2D, a first suture anchor 20 of the remaining anchors passes back anchor stop 454 and is subsequently stopped by front anchor stop 452.

Front anchor stop 452 optionally comprises a bushing 470 optionally spring loaded by a resilient element represented by a spring 471. Bushing 470 lodges against tapered front end 22 of the first suture anchor to stop forward motion of the suture anchor in feed channel 422 until drive needle 40 coupled to the suture anchor by splay spurs 43 is pushed forward to move out of feed channel 422 and drive needle jaw 420. Back anchor stop 454 comprises a leaf spring 472 that catches on back end 30 of suture anchor 20 when the back end passes the leaf spring. Back anchor stop 454 operates to prevent suture anchor 20 stopped between front anchor stop 452 and back anchor stop 454 displacing backward when drive needle 40 is retracted into drive needle jaw 420 to pick up and drive another suture anchor 20 through a region of tissue after having driven a previous suture anchor 20 through the tissue.

FIGS. 4A 8C schematically show components of control handle 202 comprised in suturing device 200, and operation of the components in performing various steps involved in securing a suture thread 60 to a region of tissue using suture anchors 20, in accordance with an embodiment of the invention.

FIG. 4A-4C schematically shows internal features of control handle 202 of suturing device 200 when alligator jaws 400 are open before grabbing a region of tissue 500, the first time to secure a suture thread to the region. Tissue region 500 is schematically shown positioned on backstop 411 of alligator backstop jaw 410.

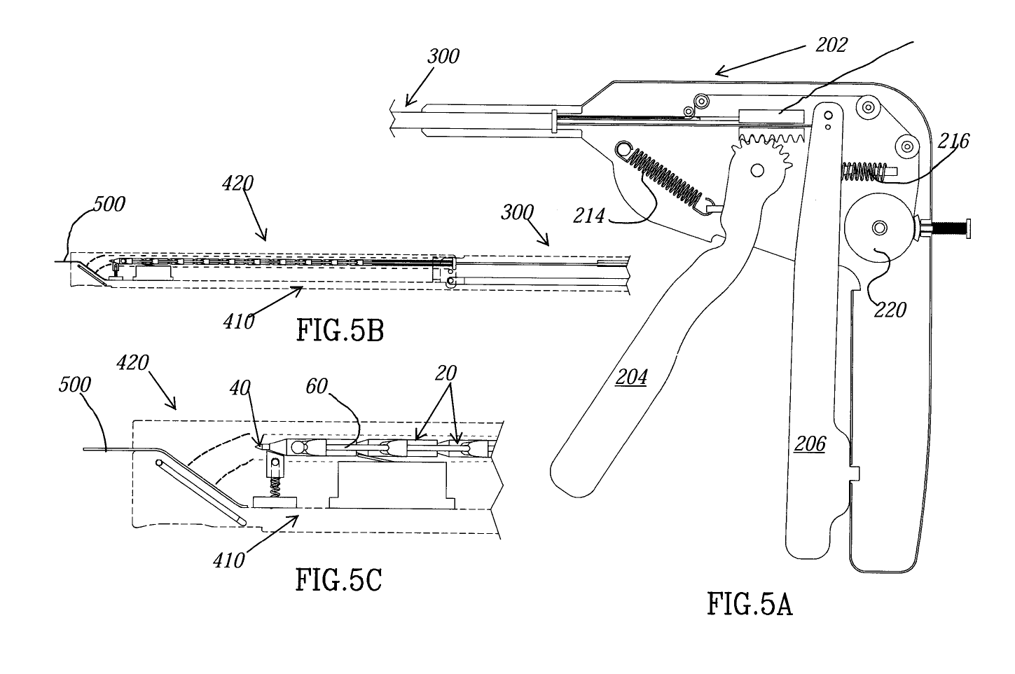

In an embodiment of the invention, trigger 204 of control handle 202 is coupled to drive needle push rod 433 by a rack and pinion arrangement in which a rack 210 is connected to drive needle push rod 433 and trigger 204 is coupled to rack 210 by a gear teeth 212. A bias spring 214 optionally rotates trigger 204 to a forward position and therefore translates rack 210 to a backward position which, unless trigger 204 is pulled, pulls drive needle push rod back and maintains drive needle 40 retracted into drive needle jaw 420. Trigger 206 is connected to push rod 426, which controls attitude of drive needle jaw 420 to control opening and closing alligator jaws 400. Optionally, a bias spring 216 pushes trigger 206 to a forward position, which unless trigger 206 is pulled, pushes push rod 426 forward, rotating drive needle jaw 420 away from backstop jaw 410 and opening the jaws. In an embodiment of the invention control handle 202 comprises a suture feed that provisions suture thread 60 to suture anchors 20 and is controllable to maintain a desired tension in the suture thread. Optionally, the suture feed comprises a reel 220 on which the suture thread is wound and a system of sheaves 222 that direct the suture thread to barrel 300 and suture anchors 20 (FIGS. 5B and 5C). In an embodiment a spring loaded brake shoe 224 is coupled to reel 220 to control tension in suture thread 60. FIGS. 4B and 4C schematically show features of suturing device 200 for the state of triggers 204 and 206 shown in FIG. 4A.

FIGS. 5A-5C schematically shows features of suturing device 200 shown in FIGS. 4A-4C when trigger 206 is pulled to rotates drive needle jaw 420 to backstop jaw 410 close the jaws to clamp and hold tissue region 500.

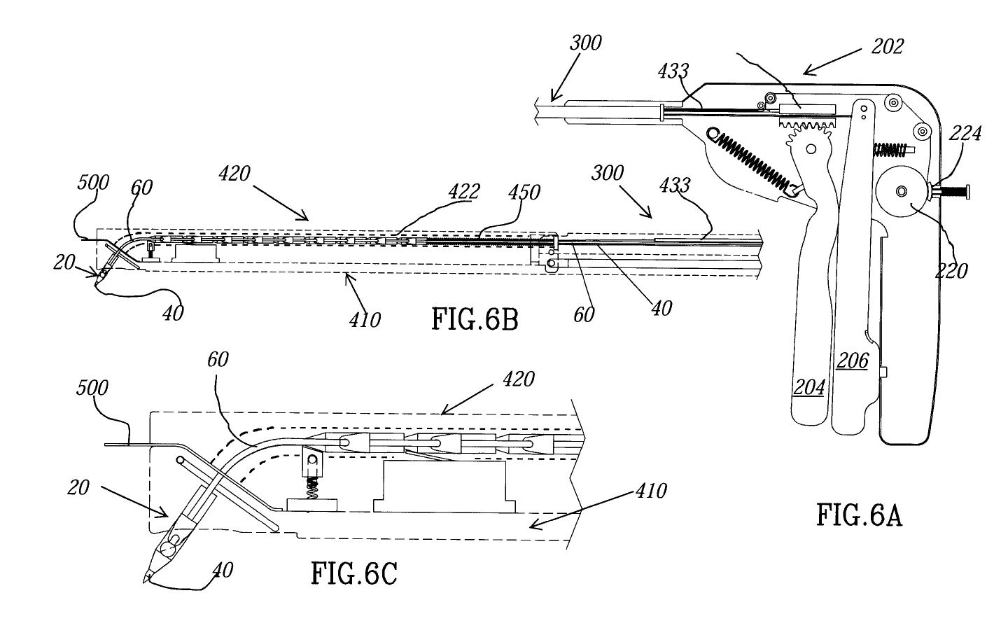

FIG. 6A schematically shows features of suturing device 200 when after trigger 206 is pulled to clamp tissue region 500, trigger 204 is pressed to translate rack 210 forward (towards support barrel 300), and thereby push drive needle push rod 433 forward to push drive needle 40 out of drive needle jaw 420 and drive a suture anchor 20 through tissue region 500. FIGS. 6B and 6C show suture drive needle 40 driven through tissue region 500.

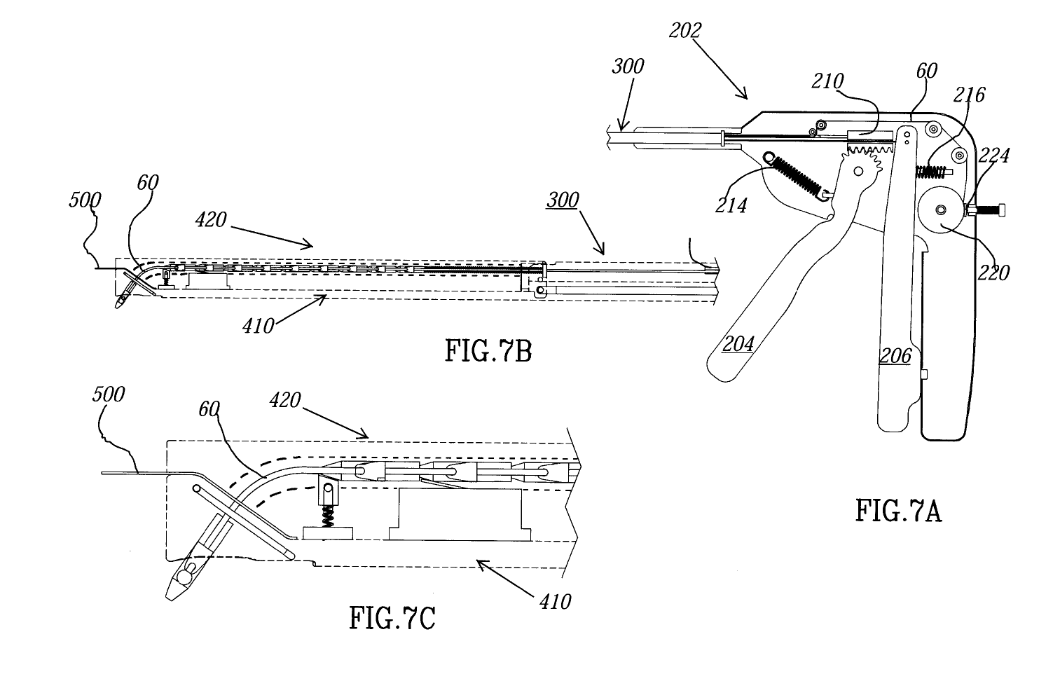

In FIG. 7A trigger 204 is released. Bias spring 214 rotates trigger 204 clockwise (in FIG. 7A) to its front position translating rack 210 backwards and extracting drive needle 40 from tissue layer 500 and suture anchor 20 driven through tissue layer 500, and retracting the drive needle into drive needle jaw 420. FIGS. 7B and 7C schematically show enlarged portions of suturing device 200 from which it may be seen that drive needle 40 present in suture anchor 20 in FIG. 6B and FIG. 6C is absent from the suture anchor in FIGS. 7B and 7C.

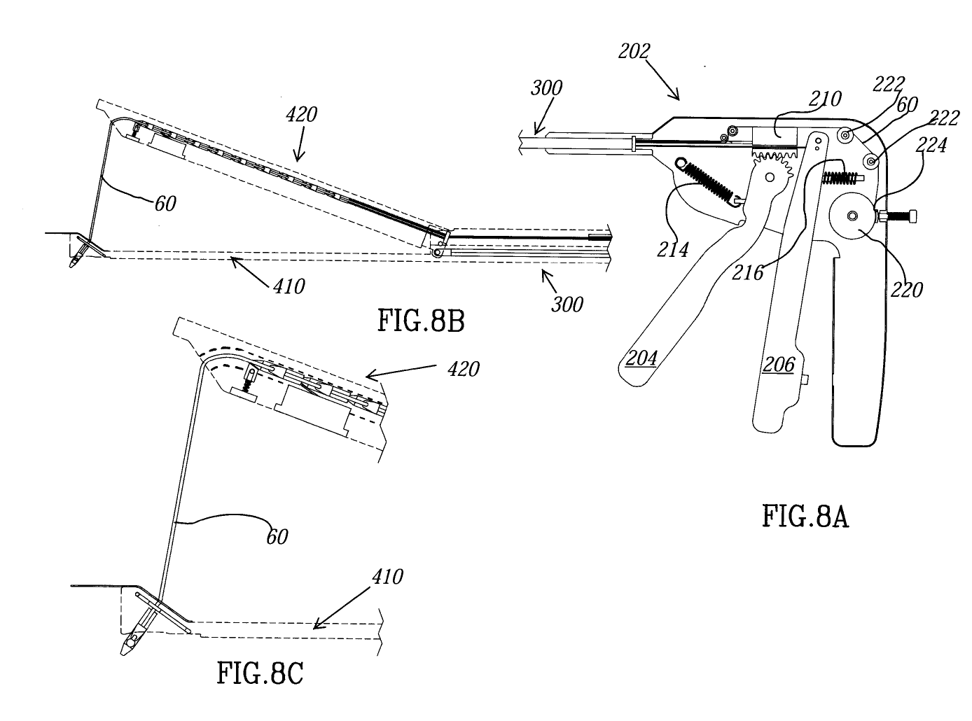

After retraction of drive needle 40 schematically illustrated in FIGS. 6A-6C trigger 206 is released and the trigger is pushed forward by bias spring 216 rotating drive needle jaw 420 away from backstop jaw 410 as schematically shown in FIG. 8A. In rotating away from backstop jaw 410, as a result of tension in suture thread 60 generated by forces applied by spring loaded brake shoe 224 to suture reel 220, drive needle jaw takes up slack in suture thread 60 between suture anchor 20 driven through tissue layer 500 and applies force to fin 28 of the suture anchor. FIGS. 8B and 8C schematically show drive needle jaw 420 rotated away from backstop jaw 210 and “pulling” suture thread 60 to take up slack in, and tension, the suture thread. The force generates a torque that rotates the suture anchor so that it is oriented with respect to tissue layer 500 similar to the orientation of suture anchor 20-1 with respect to tissue layer 70 shown in FIG. 1E.

In the description and claims of the present application, each of the verbs, “comprise” “include” and “have”, and conjugates thereof, are used to indicate that the object or objects of the verb are not necessarily a complete listing of components, elements or parts of the subject or subjects of the verb.

Descriptions of embodiments of the invention in the present application are provided by way of example and are not intended to limit the scope of the invention. The described embodiments comprise different features, not all of which are required in all embodiments of the invention. Some embodiments utilize only some of the features or possible combinations of the features. Variations of embodiments of the invention that are described, and embodiments of the invention comprising different combinations of features noted in the described embodiments, will occur to persons of the art. The scope of the invention is limited only by the claims.

1. Apparatus for securing a suture thread to a region of tissue, the apparatus comprising:
first and second elongate jaws respectively having first and second longitudinal axes and first and second gripping surfaces for gripping and holding a region of tissue at a tilt angle relative to the first and second axes when the jaws are closed;
a channel in the first elongate jaw having a substantially straight section extending along the first longitudinal axis and a curved section that extends from the straight section to the first gripping surface for housing at least one suture anchor configured to secure a portion of a suture thread to the tissue region, and;
a flexible needle inside the channel that extends along the straight section and is controllable to extract a suture anchor in the channel from the first jaw through the curved section and drive the extracted suture anchor through the tissue region to secure the suture thread.

2. The apparatus according to claim 1 wherein the tilt angle is within a range of about 30° and about 60°.

3. (canceled)

4. (canceled)

5. The apparatus according to claim 1 wherein a tangent to the curved section substantially in a region where the curved section meets the first gripping surface is substantially perpendicular the first gripping surface.

6. The apparatus according to claim 1 wherein the curved section has a minimum radius of curvature greater than about 2.5 mm.

7. (canceled)

8. The apparatus according to claim 1, wherein a cross section of the channel has a maximum dimension within a range of about 0.8 mm and about 2.5 mm.

9. (canceled)

10. The apparatus according to claim 1, wherein the second jaw is formed having a recess in the second gripping surface for receiving the suture anchor driven through the tissue region by the needle.

11. (canceled)

12. (canceled)

13. The apparatus according to claim 1, wherein the needle has a circular cross section having a diameter within a range of about 0.15 mm and about 0.6 mm.

14. (canceled)

15. The apparatus according to claim 1, further comprising a suture thread feed that provisions suture thread to suture anchors.

16. The apparatus according to claim 15 wherein the suture thread feed is controllable to maintain a desired tension in the suture thread.

17. The apparatus according to claim 1, wherein the suture anchor has an elongate body having a lumen through which the flexible needle passes.

18. (canceled)

19. The apparatus according to claim 17, wherein the elongate body is circularly cylindrical and has an external diameter within a range of about 0.3 mm and about 1.5 mm.

20. (canceled)

21. The apparatus according to claim 17, wherein the lumen is formed having first and second circularly cylindrical chambers having different diameters.

22. The apparatus according to claim 17, further comprising a protuberance on the body formed having a hole through which the suture thread passes.

23. The apparatus according to claim 17, wherein the suture anchor is formed from at least one, or any combination of materials chosen from the group of materials consisting of: Teflon, an elastomer, and silicon.

24. A suture anchor for securing a suture thread to a region of tissue comprising an elongate body having a lumen through which the flexible needle passes and a protuberance on the body formed having a hole through which the suture thread passes.

25. The suture anchor according to claim 24 wherein the elongate body has a tapered front end.

26. The suture anchor according to claim 24, wherein the elongate body is circularly cylindrical and has an external diameter greater than or about equal to 0.3 mm.

27. The suture anchor according to claim 24, wherein the elongate body is circularly cylindrical and has an external diameter less than or about equal to 1.5 mm.

28. The suture anchor according to claim 24, wherein the lumen is formed having first and second circularly cylindrical chambers having different diameters.

29. The suture anchor according to claim 24, wherein the suture anchor is formed from at least one, or any combination of materials chosen from the group of materials consisting of: Teflon, an elastomer, and silicon.
